(19) United States
(12) Plant Patent Application Publication
(10) Pub. No.: US 2024/0389489 P1
(43) Pub. Date: Nov. 21, 2024

(54) Anthurium Plant Named 'BLOCANTMIC'

(50) ;

(71) Applicant: Jeffrey Sherwood Block, Miami, FL (US)

(72) Inventor: Jeffrey Sherwood Block, Miami, FL (US)

(21) Appl. No.: 18/831,045

(22) Filed: May 15, 2024

(30) Foreign Application Priority Data
May 16, 2023 (QZ) 2023/1067

(51) Int. Cl. A01H 6/12

(52) U.S. Cl. CPC A01H 6/12
USPC PLT/365

(57) ABSTRACT

‘BLOCANTMIC’ is a new and distinctive variety of plant which is characterized by the combination of vigorous growth, dark greyed-purple petioles, a dark green mature upper foliar surface with light greyed-green veins, a very light greyed-green lower leaf surface with greyed-red foliar venation, and the stability of all characteristics. from generation to generation.

Latin name of the genus and species:

The Latin name of the genus and species of the novel variety disclosed herein is magnificum.

Variety denomination:

The inventive variety of disclosed herein has been given the variety denomination ‘BLOCANTMIC’.

### BACKGROUND OF THE INVENTION

Parentage: The new cultivar is a seedling selection which resulted from the multigenerational, controlled self-pollination of an unnamed magnificum plant (not patented) carried out in Miami, Floria. Over a period of thirty years, the inventor self-crossed the parent plant through four distinct generations. With each progeny population of approximately 250 to 500 seedling accessions, the inventor evaluated for a superior combination of improved plant vigor and unique foliage with the darkest upper leaf surface and lightest lower leaf surface with conspicuous dark red veins. In May of 2019, one progeny from the fourth generation of accessions was observed to exhibit the best combination of these characteristics. After further evaluation to confirm the unique characteristics, the new cultivar was selected in April of 2022 for commercialization and given the breeder denomination ‘BLOCANTMIC’.

Asexual Reproduction: Asexual reproduction of ‘BLOCANTMIC’, by way of meristematic tissue culture, was first performed in April of 2020 in Eustis, Florida. Multiple successive generations so produced have shown that the unique features of the instant cultivar are stable and reproduced true to type.

### SUMMARY OF THE INVENTION

The cultivar ‘BLOCANTMIC’ has not been observed under all possible environmental conditions and the phenotype may vary somewhat with variations in the instant environment such as temperature, day length, and light intensity, without, however, any variance in genotype. The following characteristics have been repeatedly observed and represent the distinguishing characteristics of the new cultivar ‘BLOCANTMIC’. These traits, in combination, distinguish ‘BLOCANTMIC’ as a new and distinct cultivar.
1. Anthurium ‘BLOCANTMIC’ is a vigorously-growing herbaceous perennial with a flattened spherical profile; and
2. Anthurium ‘BLOCANTMIC’ exhibits durable foliage borne on dark greyed-purple petioles; and
3. Anthurium ‘BLOCANTMIC’ exhibits juvenile foliage with an adaxial surface that is colored with a mixture of dark greyed-green and brown and veins that are colored greyed-red; and
4. Anthurium ‘BLOCANTMIC’ exhibits juvenile foliage with an abaxial surface that is colored a very light greyed-green and veins that are colored greyed-purple; and
5. Anthurium ‘BLOCANTMIC’ exhibits mature foliage with an adaxial surface that is colored with a mixture of dark greyed-green and yellow-green and veins that are colored with a light shade of greyed-green; and
6. Anthurium ‘BLOCANTMIC’ exhibits mature foliage with an abaxial surface that is colored a very light greyed-green and veins that are colored greyed-red.

### BRIEF DESCRIPTION OF THE FIGURES

FIG. 1 shows, as nearly true as it is reasonably possible to make the same in color illustrations of this type, an exemplary mature plant of ‘BLOCANTMIC’, grown in a greenhouse in Honselerdijk, the Netherlands. The plant shown is potted into a 21 cm nursery pot.

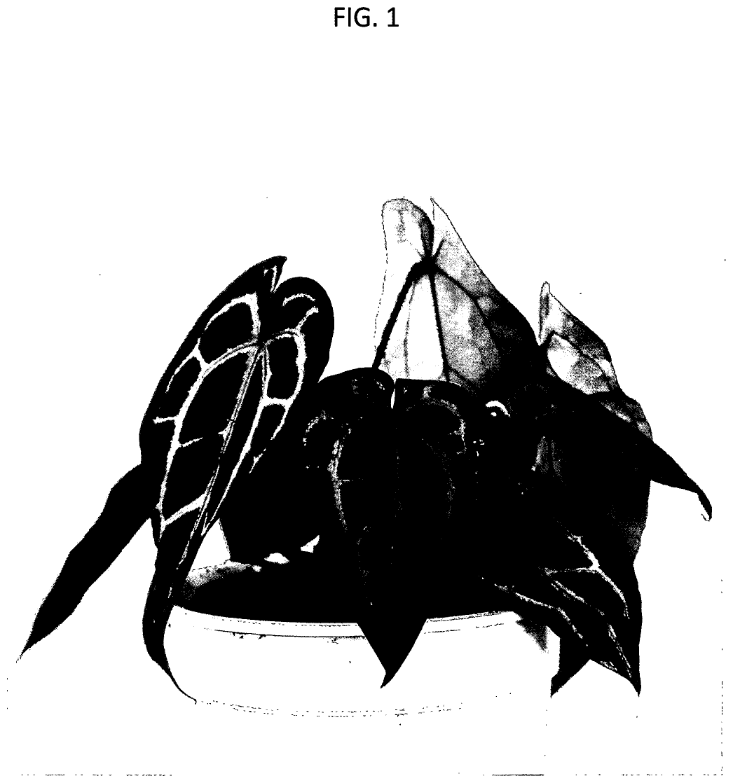

FIG. 2 shows, as nearly true as it is reasonably possible to make the same in color illustrations of this type, the typical juvenile foliage of ‘BLOCANTMIC’.

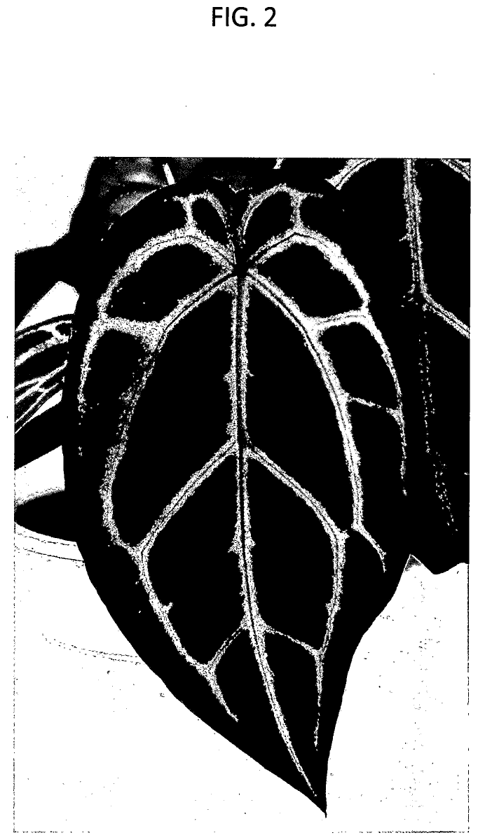

FIG. 3 shows, as nearly true as it is reasonably possible to make the same in color illustrations of this type, the typical mature foliage of ‘BLOCANTMIC’.

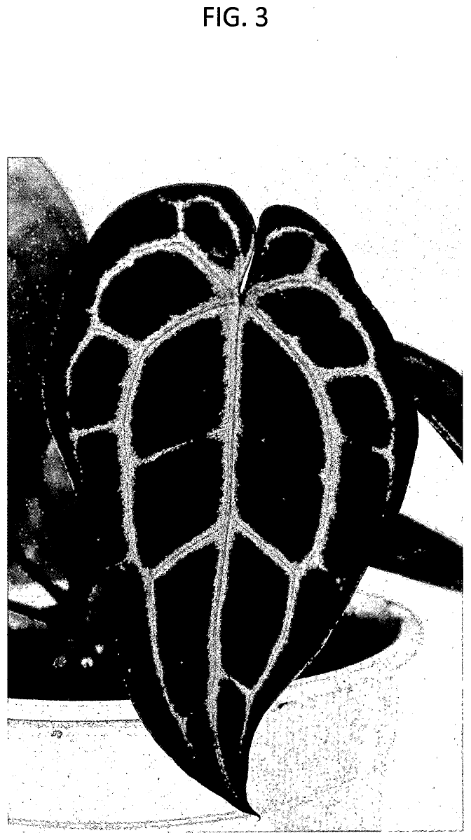

The photographs were taken using conventional techniques and although colors may appear different from actual colors due to light reflectance it is as accurate as possible by conventional photographic techniques.

### BOTANICAL DESCRIPTION OF THE PLANT

The following is a detailed botanical description of a new and distinct variety of Anthurium known as ‘BLOCANTMIC’, based upon observations made from mature plants grown in 21 cm nursery containers. Plants were grown in a greenhouse in Honselerdijk, The Netherlands and were produced using conventional greenhouse production protocols for Anthurium. Observation data was recorded in April of 2024.

Those skilled in the art will appreciate that certain characteristics will vary with older or, conversely, with younger plants. ‘BLOCANTMIC’ has not been observed under all possible environmental conditions. Where dimensions, sizes, colors and other characteristics are given, it is to be understood that such measurements are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, sixth edition.

A botanical description of ‘BLOCANTMIC’ and a comparison with the parent and most similar variety are provided below.
General plant description:
   Growth rate.—Fast-growing.
   Plant vigor.—Vigorous.
   Growth habit.—Clump-forming, herbaceous perennial.
   Plant profile shape.—Flattened spherical.
   Height.—20.9 cm in height, to the highest leaf.
   Width.—Average 34.5 cm in width.
   Propagation.—Meristematic tissue culture.
   Time to initiate roots.—Approximately 25 to 30 days to initiate roots at approximately 25 degrees Celsius.
   Environmental tolerances.—Adapt to, at least, USDA Zones 10 to 13 and temperatures ranging from as high as 40 degrees Celsius and as low as 5 degrees Celsius; moderate to high tolerance to rain; low tolerance to wind.
   Pest resistance and susceptibility.—Plants have not been observed to be susceptible or resistant to pathogens and pests common to Anthurium.
Root system:
   General.—Anthurium sp. exhibits a moderately shallow root system with fleshy adventitious roots arising from the plant crown.
   Branching.—Freely branched.
   Density.—Moderately dense.
   Distribution.—Relatively shallow.
   Texture.—Fleshy; smooth; lacking root hairs.
Stems:
   General.—Anthurium sp. is acaulescent with foliage that arises directly from the base of the plant, at the soil level, forming basal clumps; no stems or lateral branches are produced.
   Quantity of clumps per plant.—2.
Foliage:
   Arrangement.—Alternate.
   Division.—Simple.
   Quantity of leaves per lateral branch.—3 leaves per clump.
   Leaf shape.—Cordate to narrow cordate.
   Leaf apex.—Apiculate.
   Leaf base.—Cordate; lobes overlapping.
   Aspect.—Moderately to strongly concave.
   Leaf length.—Average 19.4 cm in length.
   Leaf width.—Average 11.9 cm in width.
   Leaf margin.—Entire; slightly undulate.
   Texture, adaxial surface.—Moderately to strongly bullate, glabrous, and coriaceous.
   Texture, abaxial surface.—Moderately to strongly bullate, glabrous, and coriaceous.
   Luster, adaxial surface.—Matte to slightly glossy.
   Luster, abaxial surface.—Matte.
   Juvenile color, adaxial surface.—Nearest to a mixture of greyed-green and brown, RHS N189A and 200A yet darker, and veined greyed-red, nearest to RHS 181A; areas of the lamina that are adjacent to the veins are colored grey, nearest to RHS 201D, with a conspicuous silvery sheen.
   Juvenile color, abaxial surface.—Greyed-green, nearest to RHS 197C, and veined greyed-purple, nearest to RHS 185A.
   Mature color, adaxial surface.—Nearest to a mixture of yellow-green and greyed-green, RHS 147A and N189A yet darker; areas of the lamina that are adjacent to the veins are colored black, nearest to RHS 202D, with a conspicuous silvery sheen.
   Mature color, abaxial surface.—Greyed-green, nearest to RHS 194A yet slightly darker.
   Venation.—Vein pattern — Pinnate. Mature vein color, adaxial surface — Greyed-green, RHS 197B. Mature vein color, abaxial surface — Greyed-red, nearest to in between RHS 181A and 181B.
   Petiole.—Aspect — Terete. Length — 13.4 cm, including the geniculum. Width — 0.4 cm. Texture and luster, both surfaces — Smooth, glabrous and slightly glossy. Color, adaxial surface — Greyed-purple, nearest to RHS 183A, and suffused with brown, nearest to RHS 200B. Color, abaxial surface — Greyed-purple, nearest to RHS 183A, and suffused with brown, nearest to RHS 200B. Strength — Strong. Geniculum — Length — 1.4 cm. Height — 0.35 cm. Width — 0.3 cm. Color, adaxial surface — Greyed-purple, nearest to RHS 187A. Color, abaxial surface — Greyed-purple, nearest to RHS 187B. Petiole wings — Length — Approximately 3.3 cm. Width — Approximately 0.6 cm. Color — Greyed-red, nearest to a mixture of RHS 178B and 178C.
Inflorescence: Anthurium sp. produce a spathe and spadix inflorescence. However, the inflorescence of ‘BLOCANTMIC’ has not been observed.
Seed and fruit: Seed production has not been observed.

### COMPARISONS WITH THE PARENT

Plants of the new cultivar ‘BLOCANTMIC’ differ from the parent, an unnamed Anthurium magnificum plant (not patented), in the following characteristics described in

Table 1 below.

TABLE 1

| Characteristic | ‘BLOCANTMIC’ | The parent. |
|---|---|---|
| Plant height. | Shorter than the parent. | Taller than ‘BLOCANTMIC’. |
| Foliage shape; length-to- | Cordate to narrow cordate; | Cordate to broadly cordate; |
| width ratio of the lamina. | length-to-width ratio of 1.6. | length-to-width ratio of 1.3. |
| Size of the mature foliage. | Smaller than the parent. | Larger than‘ BLOCANTMIC’. |
| Foliage texture. | Moderately to strongly bullate. | Smooth to slightly bullate. |
| General coloration of the | A mixture of greyed-purple | A mixture of yellow-green |
| petiole. | and brown. | and greyed-green. |

### COMPARISONS WITH THE MOST SIMILAR VARIETY OF COMMON KNOWLEDGE

Plants of the new cultivar ‘BLOCANTMIC’ differ from the commercial variety, Anthurium magnificum x crystallinum ‘Zara’ (not patented), in the following characteristics described in Table 2 below.

TABLE 2

| Characteristic | ‘BLOCANTMIC’ | ‘Zara’ |
|---|---|---|
| Foliage shape. | Cordate to narrowly cordate. | Cordate to broadly cordate. |
| Foliage texture. | Moderately to strongly bullate. | Strongly bullate. |
| General coloration of | A mixture of yellow-green | A mixture of yellow-green |
| the mature adaxial | and greyed-green, generally | and greyed-green, generally |
| foliar surface. | appearing as a very dark green. | appearing as a lighter shade |
| | | of green, relative to |
| | | ‘BLOCANTMIC’. |
| General coloration of the | Greyed-green. | Vivid red-purple. |
| mature foliar veins on | | |
| the adaxial surface. | | |
| General coloration | A lighter shade of greyed- | A darker shade of greyed-green, |
| of the mature abaxial | green, relative to ‘Zara’. | relative to ‘BLOCANTMIC’. |
| foliar surface. | | |

That which is claimed is:

1. A new and distinct variety of Anthurium plant named ‘BLOCANTMIC’, substantially as described and illustrated herein.
